(19) United States
(12) Patent Application Publication
(10) Pub. No.: US 2014/0068818 A1
(43) Pub. Date: Mar. 6, 2014

(54) METHOD FOR STABLE EXPRESSION OF SUPPRESSORS OF RNAI IN PLANTS BY DIRECT GENETIC TRANSFORMATION OF SEEDS

(71) Applicant: INTERNATIONAL CENTRE FOR GENETIC ENGINEERING AND BIOTECHNOLOGY, New Delhi (IN)

(72) Inventors: Neeti Sanan MISHRA, New Delhi (IN); Sudhanshu Sekhar DAS, New Delhi (IN)

(73) Assignee: INTERNATIONAL CENTRE FOR GENETIC ENGINEERING AND BIOTECHNOLOGY, New Delhi (IN)

(21) Appl. No.: 14/015,018

(22) Filed: Aug. 30, 2013

(30) Foreign Application Priority Data
Aug. 30, 2012 (IN) 2694/DEL/2012

(51) Int. Cl. C12N 15/82

(52) U.S. Cl. CPC C12N 15/8205; C12N 15/8218
USPC 800/294; 800/298

(57) ABSTRACT

The present invention relates to a method for producing transgenic plants comprising Agrobacterium mediated in planta transformation of seeds capable of overexpressing the RNAi suppressor proteins. The invention also relates to a rapid method for generating transgenic rice plants stably expressing the viral suppressors of RNAi by directly transforming the seeds and growing them in presence of appropriate selection markers. The invention provides an effective and efficient method for Agrobacterium mediated in planta transformation of rice seeds.

### FIELD OF THE INVENTION

The present invention relates to a rapid method for generating transgenics capable of overexpressing the RNAi suppressor proteins. The present invention also relates to a rapid method for generating transgenic rice plants stably expressing the viral suppressors of RNAi by directly transforming the seeds and growing them in presence of appropriate selection markers. The present invention further provides an effective and efficient method for Agrobacterium mediated in planta transformation of rice seeds.

### BACKGROUND OF THE INVENTION

The transformation of plants has emerged as a fundamental technique in the scientific understanding of plant biology and for improving existing varieties. The growth of this discipline is directed by the need to develop reproducible methods for genetic transformation of a wide range of plant species. Rice, the staple food of more than half of the world's population, has emerged as an ideal monocot model system because of its commercial value, relatively small genome size and close relationship with other important cereal crops. Great efforts have been undertaken to improve rice production via genetic engineering (Fujimoto H et al, Bio/Technology 1993, 11: 1151-1155; Paine Jacqueline A et al, Nature Biotechnology 2005, 23: 482-7).

Agrobacterium mediated transformation remains a powerful and preferred method for genetic modification of plants due to its high efficiency of transformation, integration of small numbers of copies of transfer DNA (T-DNA) into chromosomes, minimal rearrangement of transgene and transfer of relatively large segments of DNA upon transformation (Birch R G et al, Annu. Rev. Plant Physiol. Plant Mol. Bio. 1997, 48:297-326; Hansen G et al, Trends Plant Sci. 1999, 4: 226-231). The protocols being used were based on using embryonic callus cells derived from the scutella of mature seeds as the starting material and addition of acetosyringone in the co-cultivation steps (Hiei Y et al., Plant J 1994, 6:271-82; Hiei Y et al., Plant Mol Biol 1997, 35: 205-218). Since then several protocols and their modified versions are available for efficient rice transformation (Rashid H et al., Plant Cell Rep. 1996, 15: 727-730; Toki, S, Plant Mol. Biol. Rep. 1997, 15: 16-21; Yara A et al., Plant Biotech. 2001, 18: 305-310).

Even though scientific efforts have been directed to improve the transformation efficiency, the time required for obtaining embryonic calli prior to transformation and regeneration span, before a whole plant can be obtained, is quite high. Transformation techniques currently in use still present significant obstacles for the manipulation of most of the Indian rice cultivars due to low transformation competency and poor shoot regeneration potential. Even for the varieties classified as suitable for regeneration the transformation efficiencies ranging from 10-50% are not equally competent for all genes (Sallaud C et al., Theor. Appl. Genot. 2003, 106: 1396-1408; Nishimura A et al., Proc. Natl. Acad. Sci. USA 2005, 102: 11940-11944; Sahoo K K et al., Plant Methods 2011, 7:49). The transformation of genes with desired molecular characteristics is also limited by their abilities to influence shoot regeneration potential in the standard transformation procedures due to an overdose of the expressed transgene or dominant negative effect of gene products rendered non-functional. Within this category are included genes coding for the suppressors of RNAi.

RNA interference (RNAi) is a complex surveillance and regulatory process inherent to all eukaryotic cells. It mediates the post-transcriptional repression of the target gene expression and represses the proliferation and expression of different invading nucleic acids, such as viruses, viroids, transposons or transgenes. As a counter defensive mechanism, members of different viruses encode proteins known as RNA silencing suppressors that suppress RNA silencing at different stages of the pathway (Vaucheret H et al., J Cell Sci 2001,114:3083-91; Moissiard G and Voinnet O, Mol. Plant Pathol. 2004, 1: 71-82; Roth B M et al., Virus Res. 2004, 102: 97-108). Recently, these suppressors have been explored as tools to ameliorate the negative influence of RNA silencing on transgene expression in plant transformation technology (Voinnet O and Baulcombe D C, Nature 1997, 389: 553; Anandalakshmi R et al., Proc. Natl. Acad. Sci. USA 1998, 95:13079-13084; Brigneti G et al., EMBO J. 1998, 17:6739-6746; Voinnet O et al., The Plant Journal 2003, 33: 949-956). Yet, a potential problem with this approach is that the suppressors interfere with the plant regeneration potentials by interfering with normal small interfering RNA (siRNA) or micro RNA (miRNA) pathways and thus often exhibit developmental anomalies (Kasschau K D et al., Dev. Cell 2003, 4:205-217; Chapman E J et al., Genes Dev. 2004, 18:1179-1186; Dunoyer P et al., Plant Cell 2004; 16:1235-1250).

To overcome the limitations associated with the available regeneration based transformation protocols, in planta transformation was considered as an attractive alternative where mature seeds were used as the explants. This method offered convenience and broader applicability to a wide genotype range. The first in planta transformation method was reported in 1987 by Feldmann and Marks (Feldmann K A et al., Mol Gen Genet 2002, 208: 1-9). Subsequently many efficient in planta transformation methods were developed for Arabidopsis (Clough S J and Bent A F, Plant J 1998, 16:735-743), Kenaf (Kojima M et al., J Biosci Bioeng 2004, 98:136-139) and Maize (Chumakov M I et al., Russ J Genet 2006, 42:893-897). In all the crops, Agrobacterium is directed towards either the apical or axillary meristems. An in planta transformation for rice was first established in 2005 by Supartana et. al., (J Biosci Bioeng 2005, 100:391-397), by inoculating the embryonic apical mersitems of soaked seeds with Agrobacterium tumefaciens and further modified in 2009 by Lin et. al, (Plant Cell Rep. 2009, 28 (7):1065-74), where soaked mature seeds were pierced by needle and agro infected by vacuum infiltration. The present invention describes an effective and efficient in planta transformation method to introduce suppressor genes in rice seeds. The method of the present invention was successfully used to raise transgenics over-expressing the B2 protein of Flock House Virus (FHV) in rice. By employing the method disclosed in the present invention rapid recovery of desirable transgenic phenotype was possible by germinating the transformed seeds in the presence of antibiotics.

### OBJECTS OF THE INVENTION

Accordingly, it is an object of the present invention to provide simple, efficient and rapid method to raise transgenics plants overexpressing the RNAi suppressor proteins.

It is yet another object of the present invention to provide a simple and efficient in planta method to raise rice transgenics plants overexpressing the RNAi suppressor proteins.

The present invention provides rapid recovery of desirable transgenic phenotype by germinating transformed seeds in presence of appropriate selection markers.

It is also another object of the present invention to develop suppressor over-expressing transgenic plants to exploit them as molecular probes for deciphering the finer details of RNA silencing pathways and also as tools for enhancing the transgene expression in plants.

It is yet another object of the present invention to provide an efficient and effective method for Agrobacterium mediation in planta transformation of rice seeds.

### SUMMARY OF INVENTION

The present invention provides a method for producing transgenic plant expressing viral suppressors of RNAi by Agrobacterium mediated in planta transformation of seeds comprising the steps: (A) preparing vector by: (i) cloning the gene coding region under promoter and terminator with restriction sites of vector to obtain gene cascade containing promoter, gene coding region and terminator; (ii) excising and moving the gene cascade to binary vector with restriction site to obtain recombinant binary vector; (iii) selecting the recombinant binary vector by PCR amplification; (B) preparing Agrobacterium by streaking single colony of Agrobacterium harbouring the recombinant binary vector on AB medium supplemented with antibiotics; (C) sterilizing seeds by surface sterilization in ethanol; sodium hypochlorite containing Tween-20 with stirring at speed of about 50-100 rpm rinsing seeds in sterile water and drying seeds to obtain surface sterilized seeds; (D) co-cultivating and infecting seeds by: (i) inoculating colonies of Agrobacterium, of step (B) in AB minimal liquid media for overnight growth to obtain Agrobacterium culture; (ii) incubating surface sterilized seeds of step (C) with Agrobacterium culture to obtain infected seeds; (iii) drying the infected seeds and incubating on fresh MS media plates without any antibiotics for co-cultivation; (E) washing infected seeds, selecting and regenerating transgenic plants by: (i) washing Agrobacterium infected seeds in sterile water; (ii) drying the seeds and transferring to selection media for growth; (iii) incubating to obtain seedlings; (iv) transferring the seedlings to MS liquid media without sucrose for two to three days; and (v) transferring to soil pots and growing till maturity to obtain transgenic plants.

In another embodiment the present invention provides a method for producing transgenic rice plant expressing viral suppressors of RNAi by Agrobacterium mediation in planta transformation of rice seeds.

In yet another embodiment the present invention provides a method for producing transgenic plant expressing viral suppressors of RNAi by Agrobacterium mediation wherein the Agrobacterium is Agrobacterium tumefaciens strains EHA105 and LBA4404.

In still another embodiment the present invention provides a method for producing transgenic plant expressing viral suppressors of RNAi by Agrobacterium mediation wherein the gene coding region is FHVB2, the promoter is CaMV35S promoter, the terminator is NOS terminator with BamHI/SmaI restriction sites of pBI121 vector.

In another embodiment the present invention provides a method for producing transgenic plant expressing viral suppressors of RNAi by Agrobacterium mediation wherein the binary vector is pCAMBIA1300 with HindIII restriction site and contains hygromycin as plant selection marker.

In yet another embodiment the present invention provides a method for producing transgenic plant expressing viral suppressors of RNAi by Agrobacterium mediation wherein the PCR amplification is by FHVB2 forward (5′ ATG CCA AGC AAA CTC GCG 3′) (SEQ ID NO:1) and FHVB2 reverse (5′ CTA CAG TTTTGC GGG TGG GGG 3′) (SEQ ID NO:2).

In still another embodiment the present invention provides a method for producing transgenic plant expressing viral suppressors of RNAi by Agrobacterium mediation wherein the antibiotics in step (B) when Agrobacterium strain is LBA4404 is Rifampicin (25 mg/l), Streptomycin (35 mg/l) and Kanamycin (50 mg/l).

In another embodiment the present invention provides a method for producing transgenic plant expressing viral suppressors of RNAi wherein the antibiotics in step (B) when Agrobacterium strain is EHA105 is Rifampicin (25 mg/l), Chloroamphenicol (25 mg/l) and Kanamycin (50 mg/l) and kept for incubation at 28° C. for 2 days.

In yet another embodiment the present invention provides a method for producing transgenic plant expressing viral suppressors of RNAi wherein the surface sterilization in step (C) is in 20 ml of 70% ethanol for 2 minutes; in 25 ml of 2% sodium hypochlorite containing 1 drop of Tween-20 for 15 minutes with stirring at a speed of about 50-100 rpm; rinsing seeds in sterile water five to six times and then blot drying on a sterile tissue paper.

In still another embodiment the present invention provides a method for producing transgenic plant expressing viral suppressors of RNAi wherein the AB minimal liquid media in step (D)(i) is 2 ml for overnight growth at 28° C. on a rotator shaker.

In another embodiment the present invention provides a method for producing transgenic plant expressing viral suppressors of RNAi wherein the incubating in step (D)(ii) is in presence of 100 uM Acetosyringone for 6 to 18 hours duration.

In yet another embodiment the present invention provides a method for producing transgenic plant expressing viral suppressors of RNAi wherein drying in step (D)(iii) is blot drying and the incubating is for two to three days in dark under tissue culture conditions.

In still another embodiment the present invention provides a method for producing transgenic plant expressing viral suppressors of RNAi wherein in step (E)(i) the washing is two to three times in sterile water by gently swirling followed by three to four washes with an aqueous solution of Cefotoxime, 250 mg/L.

In another embodiment the present invention provides a method for producing transgenic plant expressing viral suppressors of RNAi wherein in step (E)(ii) the drying is blot drying and the selection media is MS media with 50 mg/L Hygromycin and 250 mg/L Cefotoxime.

In yet another embodiment the present invention provides a method for producing transgenic plant expressing viral suppressors of RNAi wherein in step (E)(iii) the incubating is at 29.5° C. and about 4.5 klux for three to four weeks.

In still another embodiment the present invention the method disclosed herein can be used for enhancing transgenic expression in plant.

In another embodiment the present invention provides the use of the method disclosed herein to develop suppressor over-expressing transgenic plants for deciphering the finer details of RNA silencing pathways.

In yet another embodiment the present invention provides the use of the method disclosed herein for raising transgenic over-expressing B2 protein of Flock House Virus (FHV).

### BRIEF DESCRIPTION OF THE DRAWINGS

FIG. 1: Seedlings growing at selection media (1 week) containing Hygromycin after Agrobacterium mediated in planta transformation. Wild type Pusa Basmati, plain vector pCAMBIA 1302 transgenics and FHVB2 transgenic (left to right).

FIG. 2: Genomic PCR. Agarose gel electrophoresis of PCR amplified DNA from leaf tissue of the transformed rice lines. Lane 1. 1 kb DNA ladder (Fermentas); Lane 2, untransformed control: wild type plant; Lane 3-9, represents different independent lines. The band at 321 bp represent the amplified DNA of FHVB2 gene.

FIG. 3: Southern blot analysis of transgenic plants containing FHVB2 gene. 20 μg of DNA from putative transgenic plants were digested, subjected to electrophoresis and transferred to nylon membranes. Membranes were probed with ³²P labelled FHVB2 gene. Lane 1, Template probe; Lane 2, Wild type (WT) plants; Lane 3-12: Genomic DNA of transgenic plants digested with BamHI; Lane 13-14: positive control ie 50 and 250 pg of pCAMBIA1300-B2 plasmid respectively.

FIG. 4(a): Reverse transcriptase polymerase chain reaction (RT-PCR) analysis of fhvb2 gene expression in rice transgenic lines. Total RNA was separated from leaf tissue of non-transformed and transformed rice lines. RT-PCR was performed using the primers amplifying the region of fhvb2 and β-actin gene. cDNA was prepared using 50 U of SuperScript™ II reverse transcriptase (Invitrogen). Lane 1-5, rice transgenic lines; Lane 6, wild type (WT) control; Lane 7, RT control; Lane 10, PCR control with 50 pg of plasmid as template.

FIG. 4(b): Northern blot analysis of transgenic rice plants. Total RNA (30 μg) was prepared from leaf tissue obtained from non-transformed (wild type control) and transformed rice lines. Total RNA were separated on formaldehyde gel, transferred to a nylon membrane, and hybridized with ³²P labelled full length fhvb2 probe. Lane 1, wild type control; Lane 2: 50 pg of plasmid; Lane 3-7, rice transgenic lines. Expression level of B2 RNA from Northern blot analysis was done by calculating relative band intensity with spot density measurement (Alpha-view software).

### DETAILED DESCRIPTION OF THE INVENTION

The present invention provides a method for generating transgenic plants stably expressing the viral suppressors of RNAi by directly transforming the seeds. The present invention describes an effective and efficient method for Agrobacterium mediated in planta transformation of seeds. The method of the present invention was standardised in laboratory conditions using seeds of Indian rice variety Pusa basmati and the B2 protein of Flock House Virus (FHV).

FHVB2 is a well known virus-silencing suppressor that binds long dsRNA and single stranded RNA with high affinity and prevents dicer cleavage and subsequent generation of siRNAs (Lingel A. et al., EMBO Rep 2005, 6: 1149-1155). It has been reported that viral suppressors of RNAi interfere with the biogenesis and function of miRNAs. Thus it is extremely difficult to regenerate transgenic plants expressing the suppressors of RNAi using the traditional callus culture based protocols (Siddiqui S A et al., Mol Plant Microbe Interact. 2008, 21 (2):178-87; Sunter G et al., Virology 2001, 285:59-70). The present invention specifically describes a simple and efficient method for raising transgenic rice plants expressing the viral suppressor, FHVB2, by in planta inoculation of rice seeds and allowing them to grow into seedlings ex vitro. The method of the present invention comprises:

### Vector Preparation

The coding region of the suppressor is cloned under the promoter and terminator with restriction sites of vector. The gene cassette, including the promoter, gene and terminator, is excised and moved to the binary vector with restriction site. The recombinant binary vector, contains a specific plant selection marker. The recombinant vector is mobilized into Agrobacterium strains by using PCR amplifications.

### Preparation of Agrobacterium

Single colony of Agrobacterium strain harbouring the vector is streaked on specific medium supplemented with specific antibiotics for binary vector selection.

### Preparation of Transformable Material

Freshly dehusked mature plant seeds are surface sterilized in 70% ethanol and then in 2% sodium hypochlorite that contains Tween-20 for 15 min with stirring at slow speed. The seeds were rinsed in sterile water and then blot dried on a sterile tissue paper.

### Co-Cultivation and Infection

Colonies of recombinant Agrobacterium grown on specific medium, are taken and inoculated in minimal liquid media for overnight growth at 28° C. on a rotator shaker. The Agrobacterium culture is used for inoculating aliquots of minimal media at specific concentration. The surface sterilized plant seeds may also be incubated with Agrobacterium culture across 6-18 h duration for infection. The infected plant seeds are blot dried and are placed on fresh media plates without any antibiotics for co-cultivation. These are incubated for 2-3 days in dark under tissue culture conditions.

### Washing of Infected Seeds, Selection and Regeneration of Transgenic Plants

The Agrobacterium infected seeds are washed 2-3 times in sterile water by gently swirling followed by 3-4 washes with an aqueous solution of antibiotic such as Cefotaxime. Subsequently, the seeds are blot-dried and transferred to selection media containing with antibiotics such as Hygromycin and Cefotaxime for further growth and incubated at 28 to 30° C. and ˜4.0 to 5.0 klux for 2 to 5 weeks. Seedlings (10-12 cm in length) are transferred to liquid media in culture tubes for 2-3 days and then transferred to soil pots (one seedling per pot) and grown till maturity. The plants obtained from seeds germinated on selection media exhibited near normal morphology and growth and are fertile.

In a preferred embodiment the following method hereinafter describes the method for raising rice seeds but the method can be used for raising other plant seeds using a similar vector.

### Vector Preparation

The FHVB2 coding region was cloned under the CaMV35S promoter and NOS terminator with BamHI/SmaI restriction sites of pBI121 vector. The gene cassette, including the CaMV 35S promoter, fhvb2 gene and NOS terminator, was excised and moved to the binary vector pCAMBIA1300 with HindIII/EcoRI restriction site. The recombinant binary vector, pCAMBIA1300-B2, contained hygromycin as plant selection marker.

The recombinant pCAMBIA1300-B2 plasmid was mobilized into Agrobacteriun tumefaciens strains EHA105 and LBA4404 for rice transformation. Recombinant colonies were selected and screened by colony PCR using gene specific primers. The primers fuvb2 forward (5′ ATG CCA AGC AAA CTC GCG 3′) (SEQ ID NO:1) and fuvb2 reverse (5′ CTA CAG TTT TGC GGG TGG GGG 3′) (SEQ ID NO:2) were used for PCR amplifications.

### Preparation of Agrobacterium

Single colony of Agrobacterium strain LBA4404 harbouring the pCAMBIA1300-B2 vector was streaked on AB medium (Chilton M. D et al., Proc. Natl. Acad. Sci. USA 71: 3672-3676), supplemented with Rifampicin (25 mg/l), Streptomycin (35 mg/l) and Kanamycin (50 mg/l) for binary vector selection, whereas the strain EHA105 harbouring pCAMBIA1300-B2 was streaked on AB medium with Rifampicin (25 mg/l), Chloroamphenicol (25 mg/l) and Kanamycin (50 mg/l) for binary vector selection and kept for incubation at 28° C. for 2 days.

### Preparation of Transformable Material

Freshly dehusked mature seeds were surface sterilized in 20 ml of 70% ethanol for 2 min and then in 25 ml of 2% sodium hypochlorite that contains 1 drop of Tween-20 for 15 min with stirring at a speed of about 50-100 rpm. The seeds were rinsed in sterile water five to six times and then blot dried on a sterile tissue paper.

### Co-Cultivation and Infection

Colonies of Agrobacterium, grown on AB medium, was taken and inoculated in 2 ml of AB minimal liquid media for overnight growth at 28° C. on a rotator shaker. The 2 ml Agrobacterium culture were used for inoculating 50 ml aliquots of AB minimal media at a concentration of 1 to 5%.

In preferred embodiment the cultures with OD₆₀₀=0.1-1.0 was used for infection. In another embodiment the surface sterilized seeds were incubated with Agrobacterium culture in presence of 100 uM Acetosyringone across 6-18 h duration for infection. The infected rice seeds were blot dried and were placed on fresh MS media (Duchefa Biochemie) plates without any antibiotics for co-cultivation. These were incubated for 2-3 days in dark under tissue culture conditions.

### Washing of Infected Seeds, Selection and Regeneration of Transgenic Plants

The Agrobacterium infected seeds were washed 2-3 times in sterile water by gently swirling followed by 3-4 washes with an aqueous solution of Cefotoxime (250 mg/L). Subsequently, the seeds were blot-dried and transferred to selection media containing MS media with Hygromycin (50 mg/L) and Cefotoxime (250 mg/L) for further growth and incubated at 29.5° C. and ˜4.5 klux for 3-4 weeks. Seedlings (10-12 cm in length) were transferred to MS liquid media without sucrose in culture tubes for 2-3 days and then transferred to soil pots (one seedling per pot) and grown till maturity. The plants obtained from seeds germinated on selection media exhibited near normal morphology and growth and were fertile.

TABLE 1
Time lines for the various steps involved in
generation of transgenic plants

| | | |
|---|---|---|
| Pre-culture of Agrobacterium: | 3 | days |
| Inoculation with Agrobacterium: | 16 | hours |
| Co-cultivation: | 3 | days |
| Selection of transformed seeds: | 3-4 | weeks |
| Regeneration of transgenic plants: | 3-4 | weeks |

TABLE 2
Segregation of Hygromycin B resistance in T1 progeny plants
of transgenic rice lines.

| Line | Number of T1 seedlings | | | Ratios |
|---|---|---|---|---|
| No. | Total | Resistance (R) | Sensitive (S) | (R/S) |
| T1 | 39 | 29 | 10 | 2.9 |
| T2 | 45 | 34 | 11 | 3.09 |
| T3 | 53 | 40 | 13 | 3.08 |
| T4 | 25 | 19 | 6 | 3.17 |
| T5 | 77 | 58 | 19 | 3.05 |

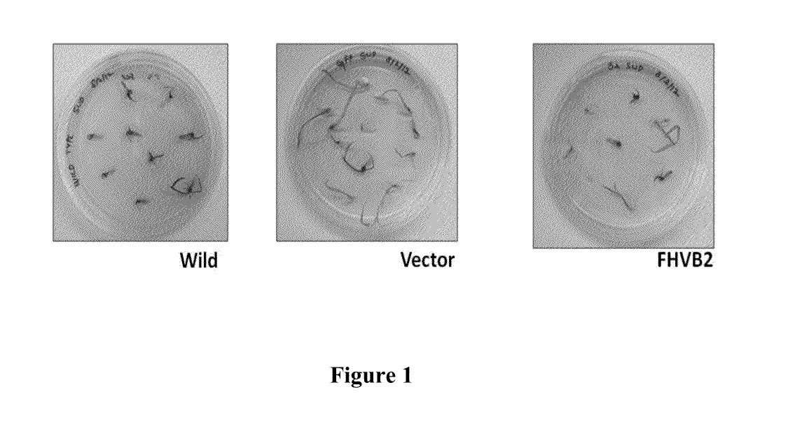

FIG. 1 shows seedlings growing at selection media (1 week) containing Hygromycin after Agrobacterium mediated in planta transformation. Wild type Pusa Basmati, plain vector pCAMBIA 1302 and FHVB2 transgenic (left to right).

### Example-1

### Genomic DNA PCR and Southern Analysis

To check for the presence of transgenes in putative transformants, genomic DNA was isolated from leaves of hygromycin-resistant and non-transformed rice plants. The genomic DNA was extracted using the CTAB (N-acetyl-N, N, N-trimethylammonium bromide) method (Murray M G and Thompson W F. Nucleic Acids Res. 1980, 8 (19):4321-5).

PCR was performed by using the primers fhvb2 forward and fhvb2 reverse. Plasmid DNA of pCAMBIA1300-FHVB2 was used as the positive control. The PCR reaction profile included initial sample denaturation at 95° C. for 5 min followed by 30 cycles of strand separation at 94° C. for 1 min, annealing at 56° C. for 30 s and extension at 72° C. for 30 s. The program ended in a final extension step for 7 min at 72° C. The amplification products of approximately 320 bp were analyzed on 0.8% agarose gel.

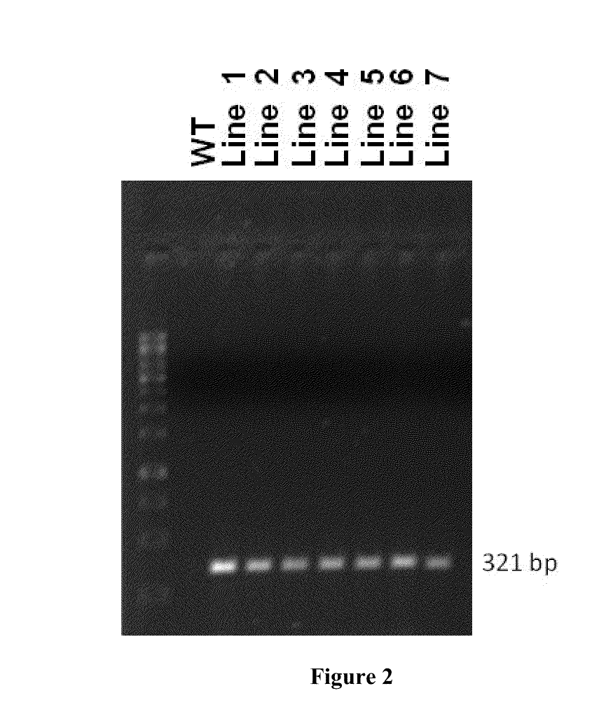

FIG. 2 shows Agarose gel electrophoresis of PCR amplified DNA from leaf tissue of the transformed rice lines. Lane 1. 1 kb DNA ladder (Fermentas); Lane 2, untransformed control wild type plant; Lane 3-9, represents different independent lines. The band at 321 bp represents the amplified DNA of FHVB2 gene.

For Southern analysis, 20 μg of genomic DNA from PCR-positive tobacco lines was digested with BamHI, electrophoresed, and blotted on Hybond N⁺ membranes (Amersham Pharmacia). α[³²P]CTP labeled 320 bp complete ORF of FHVB2 was used as a probe. The labeling was done by PCR method as described by Sambrook et al., 1989, NY., Cold Spring Harbor Laboratory Press. After hybridization for 20 h at 68° C., the membrane was washed once with 2×SSC with 0.1% SDS at 60° C. for 20 min, then washed with 0.5×SSC with 0.1% SDS at 60° C. for 30 min and finally washed with 2×SSC at room temperature before keeping it for exposure. The membrane was analyzed by phosphor-imaging.

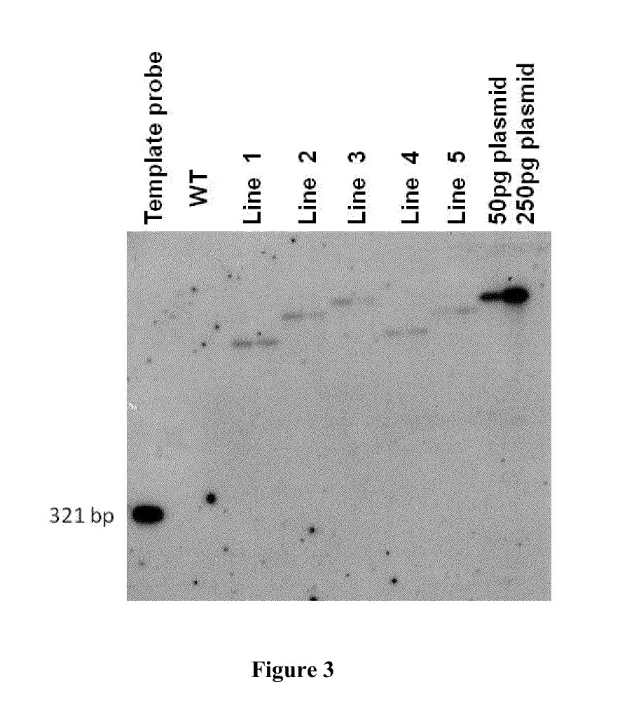

The Southern blot analysis of transgenic plants containing FHVB2 gene is shown in FIG. 3. 20 μg of DNA from putative transgenic plants were digested, subjected to electrophoresis and transferred to nylon membranes. Membranes were probed with ³²P labelled FHVB2 gene. Lane 1, Template probe; Lane 2, Wild type (WT) plants; Lane 3-12: Genomic DNA of transgenic plants digested with BamHI; Lane 13-14: positive control ie 50 and 250 pg of pCAMBIA1300-B2 plasmid respectively.

### Example-2

### Northern Blot Analysis

Total RNA was extracted from leaves of transgenic and non-transformed plants using the Guanidium thiocynate extraction method (Chomczynski, P. and Sacchi, N., Anal Biochem. 1987, 162 (1):156-9). 30 μg of total RNA were used from each plant sample was resolved on a 1.0% formaldehyde-agarose gel were transferred on Hybond N⁺ membranes (Amersham Pharmacia) and probed as described by Sambrook et al., 1989, NY., Cold Spring Harbor Laboratory Press. The probe was prepared as described above.

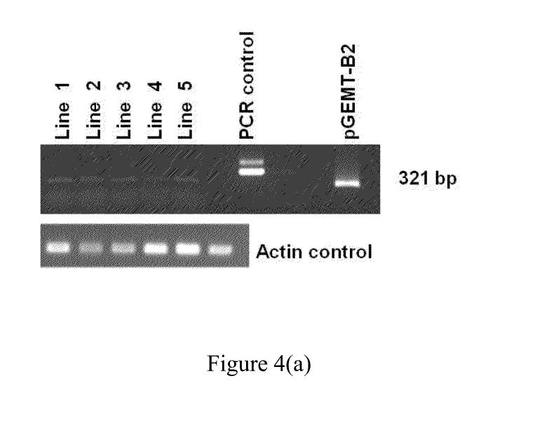

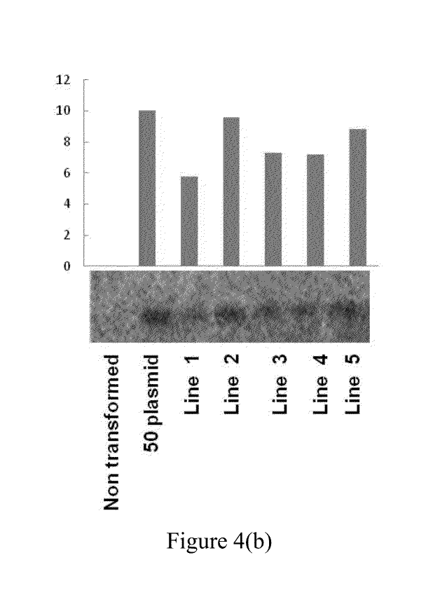

FIG. 4(b) represents Northern blot analysis of transgenic rice plants. 30 μg total RNA was prepared from leaf tissue obtained from non-transformed (wild type control) and transformed rice lines. Total RNA were separated on formaldehyde gel, transferred to a nylon membrane, and hybridized with ³²P labelled full length fhvb2 probe. Lane 1, wild type control; Lane 2: 50 pg of plasmid; Lane 3-7, rice transgenic lines. Expression level of B2 RNA from Northern blot analysis was done by calculating relative band intensity with spot density measurement (Alpha-view software).

### Example-3

### Semiquantitative PCR Analysis

For semiquantitative PCR, cDNA was prepared in 20 μL reactions from total RNA isolated from the transformed as well as non-transformed control rice plant leaf tissues, using 50 U of SuperScript™ II reverse transcriptase (Invitrogen) and random hexamers. The PCR program for the amplification of fhvb2 was carried out using the above mentioned primers at initial sample denaturation at 95° C. for 5 min followed by 30 cycles of strand separation at 94° C. for 1 min, annealing at 56° C. for 30 s and extension at 72° C. for 30 s. The program was extended for 7 min at 72° C. The rice β-actin1 gene was used as a constitutive internal standard to evaluate cDNA content. The amplification products of approximately 320 bp were analyzed on 0.8% agarose gel.

The Reverse transcriptase polymerase chain reaction (RT-PCR) analysis of fhvb2 gene expression in rice transgenic lines is shown in FIG. 4(a). Total RNA was separated from leaf tissue of non-transformed and transformed rice lines. RT PCR was performed using the primers amplifying the region of fhvb2 and β-actin gene. cDNA was prepared using 50 U of SuperScript™ II reverse transcriptase (Invitrogen). Lane 1-5, rice transgenic lines; Lane 6, wild type (WT) control; Lane 7, RT control; Lane 10, PCR control with 50 pg of plasmid as template.

### Sequences

Gene sequence: gi|22681055|ref|NC_004146.1|
(SEQ ID NO: 3)
ATGCCAAGCA AACTCGCGCT AATCCAGGAA CTTCCCGACC GCATTCAAAC GGCGGTGGAA
GCAGCCATGG
GAATGAGCTA CCAAGACGCA CCGAACAACG TGCGCAGGGA CCTCGACAAC CTGCACGCTT
GCCTAAACAA
GGCAAAACTA ACGGTAAGTC GGATGGTAAC ATCACTGCTG GAGAAACCCA GCGTGGTGGC
ATACCTAGAG
GGAAAGGCCC CCGAGGAGGC AAAACCAACA CTCGAAGAAC GCCTCCGAAA GCTGGAGCTC
GCCACAGCC
TTCCAACAAC CGGAAGTGAC CCCCCACCCG CAAAACTGTA G
Protein sequence: gi|22681058|ref|NP_689446.1|
(SEQ ID NO: 4)
MPSKLALIQE LPDRIQTAVE AAMGMSYQDA PNNVRRDLDN LHACLNKAKL TVSRMVTSLL
EKPSVVAYLE GKAPEEAKPT LEERLRKLEL SHSLPTTGSD PPPAKL

The viral suppressors of RNAi in the present invention can be used as tools to ameliorate the negative influence of RNA silencing on transgene expression in plant transformation technology. The method for stably expressing RNAi suppressors in model plant rice provided in the present invention has several advantages like:
1. It provides a successful and efficient method to generate stable transgenic lines for suppressors of RNAi bypassing the traditional callus transformation and tissue culture involving regeneration phase.
2. It applies directly to seeds, which are available throughout the year.
3. It offers convenience and applicability to broad genotype range.
4. It provides an improved in planta method which has the ability to overcome limitations of existing in planta methods as there is no requirement for vacuum infiltration or tissue injury like piercing.
5. It provides for rapid recovery of desirable transgenic phenotype as transformed seeds can be directly germinated. It takes—days to obtain a transgenic plant compare to—time normally taken by the traditional methods.
6. It provides for easy selection of desirable transgenic phenotype as transformed seeds can be directly selected by growing in presence of appropriate antibiotics.
7. 5 fold increase in transformation efficiency with a viral suppressor of RNAi when compared to the available tissue culture method.
8. Stable integration in further generation.

1. A method for producing transgenic plant expressing viral suppressors of RNAi by Agrobacterium mediation in planta transformation of seeds comprising the steps:
(A) preparing vector by:
(i) cloning the gene coding region under promoter and terminator with restriction sites of vector to obtain gene cascade containing promoter, gene coding region and terminator; (ii) excising and moving said gene cascade to binary vector with restriction site to obtain recombinant binary vector;
(iii) selecting the said recombinant binary vector by PCR amplification;
(B) preparing Agrobacterium by streaking single colony of Agrobacterium harbouring the recombinant binary vector on AB medium supplemented with antibiotics;
(C) sterilizing seeds by surface sterilization in ethanol; sodium hypochlorite containing Tween-20 with stirring at speed of about 50-100 rpm rinsing seeds in sterile water and drying seeds to obtain surface sterilized seeds;
(D) co-cultivating and infecting seeds by:
(i) inoculating colonies of Agrobacterium, of step (B) in AB minimal liquid media for overnight growth to obtain Agrobacterium culture;
(ii) incubating surface sterilized seeds of step (C) with said Agrobacterium culture to obtain infected seeds;
(iii) drying said infected seeds and incubating on fresh MS media plates without any antibiotics for co-cultivation;
(E) washing infected seeds, selecting and regenerating transgenic plants by:
(i) washing Agrobacterium infected seeds in sterile water;
(ii) drying said seeds and transferring to selection media for growth;
(iii) incubating to obtain seedlings;
(iv) transferring said seedlings to MS liquid media without sucrose for two to three days; and
(v) transferring to soil pots and growing till maturity to obtain transgenic plants.

2. The method as claimed in claim 1, wherein said transgenic plant is rice.

3. The method as claimed in claim 2, wherein said Agrobacterium is Agrobacterium tumefaciens strains EHA105 and LBA4404.

4. The method as claimed in claim 1, wherein said gene coding region is FHVB2, said promoter is CaMV35S promoter, said terminator is NOS terminator with BamHI/SmaI restriction sites of pBI121 vector.

5. The method as claimed in claim 1, wherein said binary vector is pCAMBIA1300 with HindIII restriction site and contains hygromycin as plant selection marker.

6. The method as claimed in claim 1, wherein said PCR amplification is by FHVB2 forward (5′ ATG CCA AGC AAA CTC GCG 3′) (SEQ ID NO: 1) and FHVB2 reverse (5′ CTA CAG TTT TGC GGG TGG GGG 3′) (SEQ ID NO:2).

7. The method as claimed in claim 1, wherein said antibiotics in step (B) when Agrobacterium strain is LBA4404 is Rifampicin (25 mg/l), Streptomycin (35 mg/l) and Kanamycin (50 mg/l).

8. The method as claimed in claim 1, wherein said antibiotics in step (B) when Agrobacterium strain is Rifampicin (25 mg/l), Chloroamphenicol (25 mg/l) and Kanamycin (50 mg/l) and kept for incubation at 28° C. for 2 days.

9. The method as claimed in claim 1, wherein said surface sterilization in step (C) is in 20 ml of 70% ethanol for 2 minutes; in 25 ml of 2% sodium hypochlorite containing 1 drop of Tween-20 for 15 minutes with stirring at a speed of about 50-100 rpm; rinsing seeds in sterile water five to six times and then blot drying on a sterile tissue paper.

11. The method as claimed in claim 1, wherein said AB minimal liquid media in step (D)(i) is 2 ml for overnight growth at 28° C. on a rotator shaker.

12. The method as claimed in claim 1, wherein said incubating in step (D)(ii) is in presence of 100 uM Acetosyringone for 6 to 18 hours duration.

13. The method as claimed in claim 1, wherein said drying in step (D)(iii) is blot drying and said incubating is for two to three days in dark under tissue culture conditions.

14. The method as claimed in claim 1, wherein in step (E)(i) said washing is two to three times in sterile water by gently swirling followed by three to four washes with an aqueous solution of Cefotoxime, 250 mg/L.

15. The method as claimed in claim 1, wherein in step (E)(ii) said drying is blot drying and said selection media is MS media with 50 mg/L Hygromycin and 250 mg/L Cefotoxime.

16. (canceled)

17. (canceled)

18. (canceled)

19. A transgenic plant expressing viral suppressors of RNA prepared according to the method of claim 1.


SEQUENCE LISTING (4 sequences)

SEQ ID NO: 1 - DNA, 18 bp, ARTIFICIAL SEQUENCE
  FHVB2 forward primer
atgccaagcaaactcgcg

SEQ ID NO: 2 - DNA, 21 bp, ARTIFICIAL SEQUENCE
  FHVB2 reverse primer
ctacagttttgcgggtggggg

SEQ ID NO: 3 - DNA, 320 bp, ARTIFICIAL SEQUENCE
  gi|22681055|ref|NC_004146.1|
atgccaagcaaactcgcgctaatccaggaacttcccgaccgcattcaaacggcggtggaagcagccatgggaatgagctaccaagacgcaccgaacaacgtgcgcagggacctcgacaacctgcacgcttgcctaaacaaggcaaaactaacggtaagtcggatggtaacatcactgctggagaaacccagcgtggtggcatacctagagggaaaggcccccgaggaggcaaaaccaacactcgaagaacgcctccgaaagctggagctcgccacagccttccaacaaccggaagtgaccccccacccgcaaaactgtag

SEQ ID NO: 4 - protein, 106 aa, ARTIFICIAL SEQUENCE
  gi|22681058|ref|NP_689446.1|
MPSKLALIQELPDRIQTAVEAAMGMSYQDAPNNVRRDLDNLHACLNKAKLTVSRMVTSLLEKPSVVAYLEGKAPEEAKPTLEERLRKLELSHSLPTTGSDPPPAKL